(19) United States
(12) Patent Application Publication
(10) Pub. No.: US 2009/0220473 A1
(43) Pub. Date: Sep. 3, 2009

(54) ADIR RELATED POLYMORPHISMS AND APPLICATIONS THEREOF

(75) Inventors: Cornelis Adreanus Maria Van Bergen, Den Haag (NL); Michel George Dideric Kester, Den Haag (NL); Petrus Antonius Van Veelen, Wassenaar (NL); Johan Herman Frederik Falkenburg, Oegstgeest (NL)

Correspondence Address: BROWDY AND NEIMARK, P.L.L.C.;624 NINTH STREET, NW, SUITE 300, WASHINGTON, DC, 20001-5303, US

(73) Assignee: Leiden University Medical Center, Leiden (NL)

(21) Appl. No.: 12/281,038

(22) PCT Filed: Feb. 22, 2007
(86) PCT No.: PCT/NL2007/050074

§ 371 Date: Jan. 28, 2009

(30) Foreign Application Priority Data
Feb. 28, 2006 (EP) 06110499.8

(51) Int. Cl. A61K 35/12; C12N 5/10; A61K 38/12; A61K 31/7088; C07K 14/435; C12P 21/08; C07H 21/04

(52) U.S. Cl. 424/93.71; 435/325; 514/12; 514/44 R; 530/350; 530/387.3; 530/387.9; 536/23.5

(57) ABSTRACT

The invention relates to the field of stem cell transplantations, immunotherapy and prophylaxis of neoplastic disease. Provided are peptides comprising an amino acid sequence encoded by an open reading frame as present in the nucleotide sequence of a transcript of a naturally occurring hADIR allele, wherein the amino acid sequence comprises a polymorphic MHC class I or II minor histocompatibility binding peptide.

### FIELD OF THE INVENTION

The current invention relates to the field of medicine, in particular to the fields of stem cell transplantations, immunotherapy and prophylaxis of neoplastic disease.

### BACKGROUND OF THE INVENTION

Allogeneic stem cell transplantation (SCT) is a potentially curative treatment in patients with hematological cancers^{1,2}. In addition to the anti-tumor effect of chemotherapy, antibody treatment and/or irradiation administered to the patients as the conditioning regimen prior to transplantation, an allogeneic graft versus tumor (GvT) immunoreactivity significantly contributes to the curative potential of this therapy^{3,4}. The GvT reactivity following HLA-matched SCT has been demonstrated to be mediated by T cells from the donor⁴.

Alloreactive T-cells from donor origin not only mediate the beneficial GvT effect, but are also responsible for the development of Graft versus Host Disease (GvHD) which is the major detrimental complication after allogeneic SCT⁵. T-cell depletion of the stem cell graft removes both GvHD and GvT effect^{6,7}. The anti-tumor reactivity can be reintroduced in case of relapsed hematological malignancies after transplantion by donor lymphocyte infusion (DLI). Although the postponed administration of DLI has been associated with a decreased risk of severe GvHD, both GvT and GvHD are still frequently associated in patients responding to DLI^{8,9}. Clinical observations indicate that a profound anti-tumor effect is frequently associated with GvHD, but more subtle anti-tumor reactivities can also be observed in the absence of GvHD¹⁰.

The main targets of both GvHD and GvT reactivity after HLA-matched allogeneic SCT are minor histocompatibility antigens (mHag)¹¹. Minor histocompatibility antigens (mHag) are epitopes comprised in immunogenic peptides derived from cellular proteins containing differential amino acid compositions due to polymorphisms in the genome of a subject. mHag are peptides thus differentially expressed by donor and recipient which can be recognized in the context of (self) HLA molecules. mHag may arise from differential processing of peptides due to polymorphisms in the gene encoding the protein, or by direct polymorphisms in the peptide sequence that is presented in the HLA molecules, or by differences in HLA molecules in donor and acceptor, i.e. recognition of an identical peptide in a ‘non-self’ context. Disparity in mHag between donor and recipient of allogeneic HLA-matched stem cell transplantation (SCT) leads to stimulation of mHag-specific CD4⁺ and CD8⁺ T cells that are involved in alloimmune responses, including non desirable graft rejection or graft-versus-host disease (GVHD) and desirable graft-versus-tumor (GVT) including graft-versus-leukemia/lymphoma (GVL) reactivity.

The clinical manifestations of immune responses against mHag are likely to be determined by the specific tissue expression of the proteins encoding these antigens. mHag constitutively expressed in many tissues have been suggested to be targets for combined allo-reactive GvHD and GvL responses^{12,13}. T cell responses directed against antigens that are restricted to the hematopoietic cell lineages including the malignant cells of hematopoietic origin are likely to mediate a GvT reactivity without severe GvHD¹⁴⁻¹⁸. However, also antigens that may be broadly expressed in various tissues under certain conditions, are target for a relatively specific GvT response under other circumstances^{10,19}. Induction of GVT reactivity may coincide with the development of GVHD, especially when immune responses are directed against mHags that are broadly expressed in various tissues. GVT can be separated from GVHD by induction of T cells against target structures specific for or overexpressed in tumor cells. In addition, antigens for which expression is restricted to cells of hematopoietic origin, like HA-1¹⁶, HA-2^{20,21} and BCL2A1¹⁴, may serve as specific targets for GVT. T cells specific for these antigens will destroy both malignant and normal cells of the hematopoietic system of recipient origin. Because after allogeneic SCT hematopoietic stem cells have been replaced by donor-derived cells that are not recognized by these T cells, normal donor hematopoiesis in the patient will not be affected.

The identification of tumor-associated antigens and the growing understanding of tumor-specific immune responses provide new possibilities to develop cellular immunotherapy as a strategy for the treatment of cancer. However, the results of many clinical trials have been disappointing since clinical responses were observed in only a limited number of patient^{22,23}. Vaccination protocols have not led to improvement in overall survival of cancer patients. The main impediment of these vaccination strategies is that in most cases non-mutated self-proteins were targeted. In patients, T cells specific for these self-antigens are probably anergic, tolerized or of low affinity due to peripheral or central selection processes. Therefore, characterization of mHag from patients optimally responding to cellular immunotherapeutic interventions following allogeneic SCT in the presence or absence of GvHD will lead to a better understanding of the pathogenesis of GvHD and GvL and may lead to the development of specific anti-tumor T cell therapies, antigens and medicaments.

High-avidity T cell responses capable of eradicating hematological tumors can be generated in an allogeneic setting. In hematological malignancies, allogeneic HLA-matched hematopoietic stem cell transplantation (SCT) provides a platform for allogeneic immunotherapy due to the induction of T cell-mediated graft-versus-tumor (GVT) immune responses. The clinical potency of the GVT reactivity has been demonstrated by the induction of complete remissions by administration of donor lymphocyte infusion (DLI) in patients with relapsed leukemia after allogeneic SCT^{8,9}. Immunotherapy in an allogeneic setting enables induction of effective T cell responses due to the fact that T cells of donor origin are not selected for low reactivity against self-antigens of the recipient. Therefore, high-affinity T cells against tumor- or recipient-specific antigens can be found in the T cell inoculum administered to the patient during or after SCT. The main targets of the tumor-reactive T cell responses are polymorphic proteins for which donor and recipient are disparate, designated minor histocompatibility antigens (mHag)¹⁰, or overexpressed proteins like proteinase-3.

Appropriate antigens for tumor-associated T cell responses that play a role in vivo can be identified by analysis of patients with good clinical responses after allogeneic hematopoietic SCT. Characterization of the target structures of the T cell responses in patients with relapsed hematological cancers that respond to DLI with no or limited GVHD may result in the identification of clinically relevant tumor-specific targets for immunotherapy of cancer.

Several mHag are derived from genes located at the Y chromosome (H—Y antigens) that contain polymorphic amino acids compared to their homologues encoded by the X chromosome²⁵⁻³¹ and U.S. Pat. No. 6,521,598, or have no homologue on X. These male-specific mHag have been shown play a role in sex-mismatched, HLA-matched allogeneic SCT³¹. Polymorphisms in autosomal genes have also been described to encode mHag. Some of these mHag, like HA-3¹³, HA-8¹² and UGT2B17³² display broad tissue distributions, whereas the expression of other mHags, like HA-1¹⁶ and in WO 03/047606, HA-2^{12,15}, in U.S. Pat. No. 5,770,201 and, HB-1¹⁷ and BCL2A1¹⁴, are restricted to cells of hematopoietic origin. T cell responses induced against hematopoiesis-restricted mHag may favour GVL reactivity and reduce the development of GVHD. However, it has also been described that mismatches of hematopoiesis-specific mHag, like HA-1 are correlated with GVHD, probably due a multistep development of GVHD in which T cell responses against mHag-positive antigen-presenting cells (APC) cause a local inflammation that leads to induction of T cell responses to broadly-expressed mHag.

Several mechanisms of differential expression or recognition of mHags have been described. A single nucleotide polymorphism (SNP) in the gene may result in an amino acid substitution in the protein. The polymorphism might affect a TCR contact residue as demonstrated for HB-1¹⁷ and BCL2A1¹⁴. Polymorphisms might affect splicing of the messengers or can cause changes in the antigen processing pathway, including proteasomal cleavage like demonstrated for HA-3¹³, and TAP translocation as shown for HA-8¹². Next to amino acid difference that affect antigen processing, presentation or recognition, differential mHag expression has also been described to result from deletion of a member of a multi gene family³².

The aim of the current invention is to identify new mHags with improved properties for the treatment of neoplastic disease within the context of allogeneic stem cell transplants.

### SUMMARY OF THE INVENTION

Recently, we studied in great detail a number of patients treated for relapsed hematological malignancies after allogeneic SCT with DLI. During the clinical GvT response, tumor reactive T cells were isolated based on their ability to produce Interferon-γ in response to specific activation by bone marrow containing the malignant cells. From one of the patients who was treated with DLI for relapsed multiple myeloma after transplantation with DLI and Interferon-α, we isolated a dominant T cell clone capable of recognizing the malignant multiple myeloma cells from the patient. At the time of the clinical response the patient suffered from mild GvHD, which resolved after discontinuation of Interferon and short term treatment with prednisone. The patient entered a complete remission, and is now 4 years later still in complete remission without GvHD.

Biochemical characterization of the mHAg recognized by this T cell clone revealed the antigen to be derived from a genetic polymorphism encoded by the human ATP dependent interferon responsive/Torsin3A (hADIR/TOR3A) gene. This gene was found to be highly expressed in the multiple myeloma cells, in other hematopoietic tumors as well as non-hematopoietic tumor cell lines. Recognition of normal non-malignant cells appears to be minor under steady state conditions, but activation of the target cell populations by Interferon increased recognition by the T cell clone. Based on these results it is clear that T cell responses against mHag encoded by the hADIR/TOR3A-gene may lead to a strong GvT reactivity, but also to GvHD depending on the activation state of the target tissues. The GvHD is however controllable as indicated in the case described above.

Although countless human SNPs and other polymorphisms have been identified, mHAgs are in fact quite rare and in particular only a few autosomal genes encoding mHAgs have been identified to date^{10,19}. The novel mHAg provided by the current invention is autosomal, making it more applicable than sex-bound mHAgs. Another distinct advantage of the currently identified mHAg is its relative distribution in the population, which is estimated to be around 50/50 in the Caucasian population. Such a high frequency of the polymorphism will make it easier to find compatible and matching graft-donor and graft-acceptor combinations, who are acceptable for transplantation purposes with respect to their HLA compositions, such as for stem cell transplantations (SCT) and/or DLI infusions, and yet differ in their hADIR/TOR3A allele.

The hADIR/TOR3A gene product is reported to be ubiquitously expressed, and in particular in proliferating cells and tissues substantial levels of expression are detected, making hADIR/TOR3A encoded mHAg's even more attractive candidates for eliciting immune responses to combat malignancies of hematopoietic and other origins. The interferon responsiveness of the gene makes it feasible to control and increase expression of the antigen locally or systemically, if required to boost an immune response, or to attenuate expression if the systemic immune response and/or GvHD becomes problematic.

### DETAILED DESCRIPTION OF THE INVENTION

In a first embodiment, the invention provides peptides comprising an amino acid sequence encoded by an open reading frame as present in the nucleotide sequence of a transcript of a naturally occurring hADIR/TOR3A allele, wherein the amino acid sequence comprises a polymorphic MHC class I or II minor histocompatibility binding sequence and/or peptide.

A peptide or peptide fragment according to the invention is encoded by the hADIR/TOR3A gene, the nucleic acid sequence of which is depicted in SEQ ID No. 1. The amino acid sequence of the MHC binding peptide comprises a polymorphism in one or more amino acid residues of any amino acid of SEQ ID NO: 2-5 (encoded by SEQ ID No. 1 in normal and alternative reading frames) due to a polymorphism, more preferably a single nucleotide polymorphism (SNP) in the hADIR/TOR3A gene (SEQ ID NO: 1). Preferably, the SNP encoded by the hADIR/TOR3A gene is selected from the group of SNPs currently identified in the human hADIR/TOR3A gene, including in introns (Table A: hADIR/TOR3A). In particular, changes in nucleotides 78, 672, 740, 752, and 856 in the coding exon sequence of the hADIR/TOR3A gene, are preferred for applications in the context of this invention.

Any SNP in the hADIR/TOR3A nucleic acid sequence, especially any SNP presented in Table A below, is preferably used.

TABLE A
hADIR/TOR3A SNP listing

| | | | | | | | | | | | position in (SEQ | | translation postion of SNP in codon | | | | | |
|---|---|---|---|---|---|---|---|---|---|---|---|---|---|---|---|---|---|---|
| | dbSNP | | | | | | contig | | | | ID NO: 1) | | 1 | | 2 | | 3 | |
| number | cluster id | allele | heterozygosity | validation | | | source | accession | position | function | cDNA | protein | codon | aa | codon | aa | codon | aa |
| 1 | rs 2296377 | C/T | 0.298 | 2 | 4 | 5 | mRNA | NM_022371 | 29460239 | translated | 78 | 13 | [symbol] | Leu | TCT | Ser | TTC | Phe |
| | rs17844883 | | | | | | | | | | | | [symbol] | Phe | TTT | Phe | TTT | Phe |
| | rs 17856371 | | | | | | | | | | | | | | | | | |
| | rs 17857600 | | | | | | | | | | | | | | | | | |
| | rs 17857917 | | | | | | | | | | | | | | | | | |
| | rs 17858479 | | | | | | | | | | | | | | | | | |
| 2 | rs 2296376 | T/C | 0.353 | | 1 | 2 | mRNA | NM_022371 | 29463959 | translated | 672 | 211 | [symbol] | Leu | CCT | Pro | ACC | Thr |
| | | | | | | | | | | | | | [symbol] | Leu | CTT | Leu | ACT | Thr |
| 3 | rs 12061876 | A/C | N.D. | | | | mRNA | NM_022371 | 29466044 | translated | 740 | 233 | CTT | Leu | TCT | Ser | [symbol] | Ile |
| | | | | | | | | | | | | | ATT | Ile | TAT | Tyr | [symbol] | Ile |
| 4 | rs 12092348 | A/G | N.D. | | | | mRNA | NM_022371 | 29466056 | translated | 752 | 237 | GGA | Gly | CGG | Arg | [symbol] | Ala |
| | | | | | | | | | | | | | AGA | Arg | CAG | Gln | [symbol] | Ala |
| 5 | rs 17856565 | C/T | N.D. | | | | mRNA | NM_022371 | 29466160 | translated | 856 | 272 | TCA | Ser | [symbol] | Leu | TCT | Ser |
| | | | | | | | | | | | | | CCA | Pro | [symbol] | Pro | TCC | Ser |
| 6 | rs 16853654 | C/T | 0.014 | | | | mRNA | NM_022417 | 29473507 | untransl. | 1454 | — | TAG | stop | ATA | Ile | GAT | Asp |
| | | | | | | | | | | | | | CAG | Gln | ACA | Thr | GAC | Asp |
| 7 | rs 1046385 | A/G | 0.314 | 2 | 4 | 5 | mRNA | NM_022418 | 29473814 | untransl. | 1761 | — | GGG | Gly | CGG | Arg | GCG | Ala |
| | | | | | | | | | | | | | AGG | Arg | CAG | Gln | GCA | Ala |
| 8 | rs 1128952 | C/T | 0.417 | 1 | 2 | 4 | mRNA | NM_022419 | 29474064 | untransl. | 2011 | — | CCT | Pro | TCC | Ser | TTC | Phe |
| | | | | | | | | | | | | | TCT | Ser | TTC | Phe | TTT | Phe |
| 9 | rs 2274228 | A/C | N.D. | | | | mRNA | NM_022371 | 29460941 | intron | | | | | | | | |
| 10 | rs 2274227 | A/C | N.D. | | | 4 | mRNA | NM_022371 | 29460945 | intron | | | | | | | | |
| 11 | rs 10580026 | —/ | N.D. | | | | mRNA | NM_022371 | 29461658 | intron | | | | | | | | |
| | | GGGGGGGGG | | | | | | | | | | | | | | | | |
| 12 | rs 4652355 | C/G | N.D. | | | 4 | mRNA | NM_022371 | 29461990 | intron | | | | | | | | |
| 13 | rs 12021551 | A/C | N.D. | | | 4 | mRNA | NM_022371 | 29462558 | intron | | | | | | | | |
| 14 | rs 12116579 | A/C | N.D. | | | | mRNA | NM_022371 | 29462932 | intron | | | | | | | | |
| 15 | rs 9658875 | C/T | N.D. | | | | mRNA | NM_022371 | 29463234 | intron | | | | | | | | |
| 16 | rs 912758 | G/T | 0.386 | 2 | 4 | 5 | mRNA | NM_022371 | 29463631 | intron | | | | | | | | |
| 17 | rs 12058957 | C/T | N.D. | | | | mRNA | NM_022371 | 29464112 | intron | | | | | | | | |
| 18 | rs 9659206 | A/C | 0.389 | 1 | 2 | 4 | mRNA | NM_022371 | 29464200 | intron | | | | | | | | |
| 19 | rs 10539507 | —/AA | N.D. | | | | mRNA | NM_022371 | 29464750 | intron | | | | | | | | |
| 20 | rs 9661800 | A/G | N.D. | | | | mRNA | NM_022371 | 29465609 | intron | | | | | | | | |
| 21 | rs 11589113 | A/G | N.D. | | | | mRNA | NM_022371 | 29466443 | intron | | | | | | | | |
| 22 | rs 16853640 | A/G | 0.145 | | | 2 | mRNA | NM_022371 | 29466949 | intron | | | | | | | | |
| 23 | rs 11311033 | —/A | N.D. | | | | mRNA | NM_022371 | 29467407 | intron | | | | | | | | |
| 24 | rs 12121752 | C/T | N.D. | | | | mRNA | NM_022371 | 29467814 | intron | | | | | | | | |
| 25 | rs 11811569 | A/G | N.D. | | | | mRNA | NM_022371 | 29468655 | intron | | | | | | | | |
| 26 | rs 11811579 | A/G | N.D. | | | | mRNA | NM_022371 | 29468762 | intron | | | | | | | | |
| 27 | rs 877763 | C/T | N.D. | | | 4 | mRNA | NM_022371 | 29469055 | intron | | | | | | | | |
| 28 | rs 9730271 | A/G | N.D. | | | 4 | mRNA | NM_022371 | 29469332 | intron | | | | | | | | |
| 29 | rs 1570807 | C/T | N.D. | | | 4 | mRNA | NM_022371 | 29470047 | intron | | | | | | | | |
| 30 | rs 10798652 | C/T | N.D. | | | 4 | mRNA | NM_022371 | 29470366 | intron | | | | | | | | |
| 31 | rs 4652356 | C/G | N.D. | | | | mRNA | NM_022371 | 29470599 | intron | | | | | | | | |
| 32 | rs 10158768 | G/T | N.D. | | | 4 | mRNA | NM_022371 | 29470801 | intron | | | | | | | | |
| 33 | rs 875863 | C/T | N.D. | | | | mRNA | NM_022371 | 29471224 | intron | | | | | | | | |
| 34 | rs 16853647 | A/G | 0.405 | | | 2 | mRNA | NM_022371 | 29471436 | intron | | | | | | | | |
| 35 | rs 10458367 | C/T | N.D. | | | 4 | genomic | NM_004487 | 29458545 | locus | | | | | | | | |
| 36 | rs 12124177 | C/T | N.D. | | | | genomic | NM_004487 | 29459874 | locus | | | | | | | | |
| 37 | rs 12143004 | C/T | N.D. | | | | genomic | NM_004487 | 29474502 | locus | | | | | | | | |
| 38 | rs 3813639 | C/T | 0.285 | | 2 | 5 | genomic | NM_004487 | 29459417 | locus | | | | | | | | |
| 39 | rs 3813640 | A/C | 0.302 | | 2 | 5 | genomic | NM_004487 | 29459532 | locus | | | | | | | | |
| 40 | rs 4381147 | A/G | N.D. | | | | genomic | NM_004487 | 29458172 | locus | | | | | | | | |

Bold typed codons: used in ADIR1 normal ORF

### Validation Legend of Table A:

1 Validated by multiple, independent submissions to the refSNP cluster
2 Validated by frequency or genotype data: minor alleles observed in at least two chromosomes.
3 Validated by submitter confirmation
4 All alleles have been observed in at least two chromosomes apiece
5 Genotyped by HapMap project

A peptide of the invention is normally about 8 to 12 amino acids long, small enough for a direct fit in an HLA molecule, but it may also be larger, between 12 to more than 50 amino acids and presented by HLA molecules only after cellular uptake and intra cellular processing by the proteasome and transport before presentation in the groove of an MHC molecule. The peptide may also be N- and/or C-terminally capped or modified to prevent degradation, increase stability or uptake. An mHag comprising peptide according to this invention preferably comprises the gene product of a single nucleotide polymorphism (SNP). The SNP may be comprised in the coding regions or exons of hADIR/TOR3A, or may be located in intronic sequences, affecting splicing or affecting cryptic messengers and alternative translation products as indicated in the example section. The peptide according to the invention may be encoded by any reading frame encoded by the hADIR/TOR3A gene as depicted in the amino acid sequences of SEQ ID NO: 2-5 (the hADIR/TOR3A gene product). SEQ ID NO: 2 and SEQ ID NO: 3 depict the normal reading frame (+3 frame; without or with amino acids preceding the ATG translation start codon, respectively). SEQ ID NO: 4 depicts the alternative +2 reading frame and SEQ ID NO: 5 the alternative +1 reading frame of SEQ ID NO: 1. The alternative reading frames in the hADIR/TOR3A gene, i.e. the +1 frame and the +2 frame contain many alternative start sites for transcription and translation and yield cryptic translation products. The invention demonstrates that also in these alternative reading frames and translation products, mHAg's will be present, generated by hADIR/TOR3A encoded polymorphisms. Thus, in one embodiment of the invention a peptide comprising or consisting of at least 8, 9, 10, 11, 12, 13, 14, 15 or more consecutive amino acids of SEQ ID NO: 2-5 is provided, whereby the peptide-encoding nucleic acid sequence comprises at least one SNP (preferably a SNP of Table A).

In a particular embodiment, the peptide of the invention is a peptide capable of binding an MHC molecule, and the peptide of the invention may be in the context of an MHC class I or an MHC class II molecule. One of the peptides according to the invention is designated as LB-ADIR-1F. This peptide comprises or consists of the amino acid sequence SVAPALALFPA (amino acids 18-28 of SEQ ID NO: 4), wherein the Ser at position 26 is replaced by the amino acid Phe, due to a SNP at nucleotide 78 of the hADIR/TOR3A gene (SEQ ID NO: 1). It is important to point out that for use according to this invention, whether a given polymorphism results in a polymorphic hADIR/TOR3A encoded mHag and is useful for raising an immune response, graft vs. leukemia or graft vs. tumor response, will depend on the genetic makeup and in particular HLA isotypes of both a graft-donor and graft-acceptor/recipient and their respective differences in MHC make-up. Even in the particular situation where donor and recipient are identical with respect to their hADIR/TOR3A alleles, but differ in their HLA isotypes, an immune response may arise if T cells recognize a self antigen in the context of a different HLA allele (i.e. as a ‘non-self’-configuration) as foreign antigen. Application of such antigenic reactions against hADIR/TOR3A is still within the scope of the current invention.

The mHAg containing peptides according to this invention may be comprised, used or applied in the context of an MHC class I or MHC class II molecule, for instance for raising or enhancing an T cell immune response, in order to select for binding or interacting T cell receptors, isolate or clone said T cell receptors or alternatively immunization and selection of antibodies capable of binding the mHag's and peptides of the invention, optionally in the context of a certain HLA isotype molecule.

In another embodiment, the invention provides nucleic acid molecules encoding the peptide comprising hADIR/TOR3A polymorphisms and mHAg's according to invention. These nucleic acids may be useful as means for producing the peptides of the invention or alternatively as pharmaceutical compositions or DNA vaccines, to elicit, accelerate, prolong or enhance an immune response, in particular a desirable graft vs. tumor response in a subject. In one embodiment the subject may be graft-donor, in another embodiment the subject may be the graft-recipient. Preferably, the nucleic acids of the invention may be comprised in a nucleic acid vector, such as a plasmid, cosmid, an RNA or DNA phage or virus, or any other replicable nucleic acid molecule, and are most preferably operably linked to regulatory sequences such as (regulatable) promoters, initiators, terminators and/or enhancers.

In another embodiment, the current invention provides T cell receptor (TCR) molecules capable of interacting with the hADIR/TOR3A polymorphism encoded mHags containing peptides and in particular nucleic acid molecules encoding such a T cell receptor, optionally comprised within a nucleic acid vector for expression and/or cloning purposes. A TCR according to this invention will preferably be capable of interacting with the hADIR/TOR3A encoded polymorphic mHAg's comprising peptides when they are in the context of and/or displayed by an HLA molecule, preferably on a living cell in vitro or in vivo. T cell receptors and in particular nucleic acids encoding TCR's according to the invention may for instance be applied to transfer a TCR from one T cell to another T cell and generate new T cell clones. By this TCR cloning method, T cell clones may be provided that essentially are of the genetic make-up of an allogeneic donor, for instance a donor of lymphocytes. The method to provide T cell clones capable of recognizing an mHag comprising peptide according to the invention may be generated for and can be specifically targeted to tumor cells expressing a human hADIR/TOR3A polymorphic mHag in a graft recipient, preferably a SCT and/or DLI recipient subject. Hence the invention provides T lymphocytes encoding and expressing a T cell receptor capable of interacting with a polymorphic mHag encoded by a reading frame in hADIR/TOR3A gene, preferably in the context of an HLA molecule. Said T lymphocyte may be a recombinant or a naturally selected T lymphocyte. T lymphocytes of the invention may also be used for or in the methods and pharmaceutical compositions of the invention. This specification thus provides at least two methods for producing a cytotoxic T lymphocyte of the invention, comprising the step of bringing undifferentiated lymphocytes into contact with a polymorphic hADIR/TOR3A minor histocompatibility antigen under conditions conducive of triggering an immune response, which may be done in vitro or in vivo for instance in a patient receiving a graft, using peptides according to the invention. Alternatively, it may be carried out in vitro by cloning a gene encoding the TCR specific for interacting with a polymorphic hADIR/TOR3A minor histocompatibility antigen, which may be obtained from a cell obtained from the previous method or from a subject exhibiting an immune response against an hADIR/TOR3A mHAg, into a host cell and/or a host lymphocyte obtained from a graft recipient or graft donor, and optionally differentiate to cytotoxic T lymphocyte (CTL).

In yet another embodiment, the invention provides new means; pharmaceuticals and/or medicaments, to treat malignancies expressing the hADIR/TOR3A protein. The medicament is to be administered to a patient or subject suffering from a malignancy in an amount sufficient to at least reduce the growth of the malignancy, preferably reduce the malignancy in size and most preferably eradicates the malignancy. The patient or subject to be treated preferably is a human, and in a preferred embodiment a human subject undergoing a transplant such as a SCT. The malignancies to be treated according to this invention may be any neoplastic disease expressing hADIR/TOR3A, comprising all hematological malignancies such as leukemia's, lymphoma's and (multiple) myeloma's, and all solid tumors, ranging from (benign) adenoma's and polyps to invasive and/or metastatic carcinoma's. Solid tumors expressing hADIR/TOR3A are also particularly suitable for treatment according to this invention.

The methods and means of the invention are particularly suitable to be applied in the context of a subject that has undergone an allogeneic stem cell transplant, in for instance a hematopoietic stem cell transplant (SCT) or donor lymphocyte infusion (DLI), optionally after having received chemotherapy, radiotherapy or other anti-cancer treatment. The transplant is preferably, but not necessarily, HLA matched and comprises a graft obtained from an allogeneic graft donor which does not comprise at least one hADIR/TOR3A allele that is present in the recipient of the transplant or graft, and therefore seen as ‘foreign’ or ‘non-self’ by graft originating lymphocytes. Alternatively, donor and recipient may have identical hADIR/TOR3A alleles and are HLA mismatched, whereby the HLA mismatch is capable of inducing an hADIR/TOR3A specific graft vs. tumor response by presenting hADIR/TOR3A peptides in a different HLA context, recognized by the graft derived T-cells as ‘non-self’ antigen. Genotyping of donor and recipient subjects for HLA or hADIR/TOR3A alleles is a routine procedure that can be carried out by any skilled artisan using any of several standard, textbook techniques such as but not limiting to: DNA sequencing, allele specific PCR techniques, optionally combined with restriction analysis, NASBA, DNA fingerprinting or RFLP analysis or assays using allele specific antibodies.

The peptides according to the invention, which as defined before comprise an hADIR/TOR3A encoded polymorphic mHag, or lymphocytes carrying a T cell receptor capable of interacting with the mHAgs and peptides of the invention in the context of an HLA molecule, may be used for the manufacture of pharmaceutical compositions and medicaments for the treatment of subjects suffering from malignancies expressing hADIR/TOR3A. The pharmaceutical compositions according to the invention will help to elicit, accelerate, enhance or prolong an effective immune response in the subject to be treated, in particular a desirable graft versus tumor T cell immune response. A graft vs. tumor response is in particular suitable for removal of minimal residual disease or metastases after chemotherapy of hematological cancers or after radiotherapy, chemotherapy or surgical resection in the case of operable solid tumors. A graft vs. tumor response is preferably a graft vs. hematological cancer response. A graft versus tumor response against solid tumors is preferably applied to those tumors in organs or tissues which are dispensable or replaceable, and which may be completely eradicated by the graft vs. host and/or graft vs. tumor immune response without serious adverse consequences. Such organs or tissues comprise testes, kidneys, ovaria, breastglands/tissues, prostate, thyroid, cervix, uterus, bone marrow and pancreas. In a particular embodiment, the method and the medicaments of the invention may be combined with the administration or induction of Interferon, such as Interferon gamma and in particular type I interferons such as Interferon alpha and Interferon beta. These Interferons will induce the expression of hADIR/TOR3A in the subject treated and thereby help to initiate or to enhance the immune response against the mHAg by increasing the antigen levels. The invention may be used as a primary method of treatment or as an adjuvant or follow-up treatment.

In a particular embodiment, the graft (stem)cells, in particular bone marrow/lymphocyte stem cells, may be primed prior to harvesting the transplant in the donor, by bringing them into contact with the hADIR/TOR3A mHag's containing peptides or protein and/or pharmaceutical compositions according to the invention, in order to initiate, stimulate, enhance or accelerate an anti-tumor immune response against the hADIR/TOR3A mHag displaying tumor cells, after transplantation of the graft to the recipient.

The medicaments and pharmaceutical compositions according to the invention may be formulated using generally known and pharmaceutically acceptable excipients customary in the art and for instance described in Remington, The Science and Practice of Pharmacy, 21^{nd} Edition, 2005, University of Sciences in Philadelphia. In particular immune modulating compounds and adjuvants may be suitably selected and applied by the skilled artisan, such as immune modulators described in Current Protocols in Immunology, Wiley Interscience 2004.

In yet another embodiment the invention provides for antibodies, preferably human or humanized antibody, or a fragment thereof, specific for a polymorphic hADIR/TOR3A minor histocompatibility antigen, the antigen optionally being in the context of an HLA molecule. Antibodies according to the invention may be used for therapeutic and pharmaceutical purposes and aiding in an anti-tumor immune response but may also be used for diagnostic purposes, in order to monitor tumors or tumor cells whether hADIR/TOR3A mHag is displayed by these cells, or which polymorphic hADIR/TOR3A mHags are expressed and/or displayed in a (tumor-)sample, tissue or organ of subject. An antibody according to the invention is preferably capable of binding to or interacting with polymorphic hADIR/TOR3A peptides, optionally in the context of an HLA molecule. The antibody may also be an antibody raised in any other mammal, which may be humanized using conventional techniques. The antibody of the invention may be directly or indirectly labeled using conventional techniques. Suitable labels comprise fluorescent moieties (such as; GFP, FITC, TRITC, Rhodamine), enzymes (such as peroxidase, alkaline phosphatase), radioactive labels (³²P, ³⁵S, ¹²⁵I and others), immunogenic or other haptens or tags (biotin, digoxigenin, HA, 6His, LexA, Myc and others).

The antibodies and the peptides according to this invention may also be used to monitor graft anti-tumor responses by means of tetramer staining or cytokine responses, such as the induction of interleukins and/or IFN-γ.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### FIGURE LEGENDS

FIG. 1

Recognition pattern of CTL clone RDR2 on MM cells and normal hematopoietic cells. Recognition by RDR2 (closed bars) and control alloA2 CTL (open bars) was tested in CFSE based cytotoxicity assays and by IFN-γ secretion. Heterogeneous cell samples were incubated with CTL's in a 1:1 E:T ratio for 4 h. Patient bone marrow cells were counterstained with CD138 antibodies for detection of MM cells and with CD3 antibodies for detection of T cells. Patient derived MM cells were strongly lysed by RDR2 whereas patient derived T cells were weakly recognized. Both EBV LCL and PHA blasts were strongly lysed (a). PBMNC from 3 normal mHag positive donors were counterstained with different lineage specific markers. Lysis by RDR2 was significantly diminished in both B cells (p=0.02) and T cells (p=0.00004) as compared to lysis by alloA2 CTL (b). Stimulation of CTL was measured by INF-γrelease after 24 h of coculture. RDR2 stimulation by resting PBMNC subpopulations was low as compared to alloA2 CTL stimulation whereas activated B cells (EBV LCL) induced similar IFN-γ release in both CTL (c).

FIG. 2

Identification of ADIR as the polymorphic gene responsible for RDR2 recognition. Blast searching of SVAPAXAXFPA against a translated EMBL database revealed 100% identity to amino acid 13-23 from an alternative ORF of the ADIR gene. A known SNP at nt 78 results in an amino acid change from S to F (a). Peptides SVAPALAL-F-PA (closed squares) and SVAPALAL-S-PA (open squares) were synthesized and tested for RDR2 reactivity on T2 cells in a ⁵¹Cr release assay. Only cells loaded with the SVAPALAL-F-PA peptide, but not cells loaded with the SVAPALAL-S-PA peptide were lysed (b). Constructs containing patient derived DNA were generated. The start of each construct was varied to obtain translation at the start of the transcript and at both the normal and the alternative ORF. Constructs were transiently transfected into Hela-A2 cells. RDR2 was cocultured for 24 h and IFN-γ release in supernatants was measured by elisa. RDR2 stimulation was observed in all cases. Stimulation by constructs containing only the normal ORF startcodon and lacking the alternative ORF startcodon showed strongly diminished CTL recognition (c). Similar constructs containing the donor derived DNA were not recognized by RDR2 (data not shown).

FIG. 3

Tetramer staining and clonal analysis of LB-ADIR-1F specific CTL in the patient. PBMNC from the patient taken at several time points were stained with tetramer LB-ADIR-1F. Positive cells in the 7 weeks post DLI sample were single well sorted and expanded (a). TCRBV sequence analysis was performed on 44 reactive clones revealing TCRBV7S1 in 43 clones and TCRBV6S4 in 1 clone(b). Reanalysis of the patient sample was performed using counterstaining with TCRBV7 confirming a low percentage of TCRBV7 negative cells in the LB-ADIR-1F positive population (c). Reactivity of TCRBV7S1 (closed squares) and TCRBV6S4 (open triangles) expressing clones was determined using ⁵¹Cr release assays on peptide pulsed T2 cells (d) and EBV LCL cells (e), demonstrating that TCRBV6S4 expressing T cells displayed lower cytotoxicity.

FIG. 4

ADIR gene expression and modulation of recognition.

Recognition of MNC from 3 LB-ADIR-1F positive donors was measured by direct cytotoxicity in 4 h CFSE assays (a) and by 24 h IFN-γ release (b) following preincubation in medium alone (open bars) or in medium containing 1000 IU/ml IFN-α for 48 h (closed bars). Maximal peptide loading was obtained by exogenous pulsing of MNC with saturating concentrations of synthetic peptide (grey bars). IFN-α enhanced recognition of MNC as measured by direct cytotoxicity and by cytokine release. LB-ADIR-1F positive MSC's from were growth arrested during 2 days by serum deprivation and subsequently used as target cells in ⁵¹Cr release assays. Cytotoxicity was measured after 4 h and after prolonged incubation of 20 h (c). Lysis of MSC was low as compared to lysis of EBV LCL. Growth arresting of the MSC further decreased recognition.

FIG. 5

Recognition of LB-ADIR-1F positive HLA-A2 MM cells, leukemic blasts and solid tumor cell lines. Lysis of LB-ADIR-1F expressing MM cells in heterogeneous bone marrow samples was measured using the CFSE assay, leukemic blast cell populations and solid tumor cell lines using ⁵¹Cr release assays. Recognition by RDR2 is shown by closed bars and alloA2 control CTL by open bars. On the y-axis malignant cell type and the SNP at nucleotide 78 of the ADIR gene are depicted. MM and leukemic cells expressing the LB-ADIR-1F epitope (CT or TT) were recognized whereas LB-ADIR-1F negative (CC) targets were not lysed (a). HLA-A2 positive LB-ADIR-1F expressing solid tumor cell lines were also recognized (b).

### EXAMPLES

### Material and Methods

### CTL Generation and Culture

The HLA-A2 restricted mHag-specific CTL clone RDR2 was previously isolated using the IFN-y secretion assay from a peripheral blood sample of a patient at the time of clinical response to DLI as treatment for relapsed MM after SCT (Kloosterboer et al. Leukemia, 2005, 19: 83-90). CTL clones RDR2 and the allo HLA-A2 control clone MBM13 were expanded by stimulation with irradiated (50Gy) allogeneic PBMNC and patient derived EBV transformed B cells (EBV LCL) in Iscove's Modified Dulbecco's Medium (IMDM) (Cambrex, Verviers, Belgium) supplemented with penicillin-streptomycin (Cambrex), 3 mM L-glutamin (Cambrex), 5% fetal bovine serum (FBS) (Cambrex), 5% pooled human serum, 100 U/ml IL2 (Chiron, Amsterdam, The Netherlands) and 0.8 μg/ml phytohaemagglutinin (PHA) (Remel, Dartford, UK).

### Target Cell Populations

Recognition of target cells was measured in cytotoxicity assays, and stimulation of responder cells using INF-γ secretion. Various cell populations were used in both assays. PHA blasts were generated from PBMNC's by stimulation with 0.8 μg/ml PHA and subsequent culturing in IMDM supplemented with 100 U/ml IL2 and 10% FBS. EBV LCL were cultured in IMDM supplemented with 10% FBS. The HLA-A2⁺ lymphoblastoid processing defective cell line T2 (Alexander et al. Immunogenetics 1989; 29:380-388) was cultured in IMDM supplemented with 10% FBS. Hela/A2 was generated by retroviral transduction of HLA-A*0201 in LZRS in Hela Tk-cells and cultured in IMDM supplemented with 10% FBS. Adherent solid tumor cell lines TT, Brown, MCF7 and Caski were cultured in RPMI supplemented with 10% FBS. Mesenchymal cells were generated from bone marrow cells by culturing adherent cells in low glucose Dulbecco's Modified Eagle Medium (DMEM) (Invitrogen, Paysley, Scotland) supplemented with 10% FBS (Noort et al., 2002, J. Exp. Med. 30:870-878). Interferon modulation of stimulatorcells was performed by addition of IFN-α 2a (Roche, Woerden, The Netherlands).

### Cytotoxicity Assay

To determine CTL induced population specific cytotoxicity in a heterogonous target cell population we performed a CFSE-based cellular cytotoxicity assay as described before (Jedema I et al. Blood. 2004 April 103(7)). Briefly, bone marrow cells or peripheral blood cells were labelled with 2.5 μM CFSE (Molecular Probes, Leiden, The Netherlands) and incubated with CTL clones in a 1:1 ratio. After 4, 24 and 48 h, specific cell populations were counterstained with PE or APC labelled CD138, CD4, CD8, CD14 or CD19 antibodies (Becton Dickinson, Erembodegem-Aalst, Belgium). Propodium iodide was added to exclude dead cells. To allow quantification of the surviving cell numbers in each sample, 104 Flow-count Fluorospheres (Coulter Corporation, Miami, USA) were added immediately before flowcytometric analysis.

### ⁵¹Cr Release Assay

Cytotoxicity of CTL clones in standard ⁵¹Cr release assays was performed as described previously (Faber et al. 1992, J Exp Med 176: 1283-1289). Target cells were labelled with 100 μCi Na₂⁵¹CrO₄ (Amersham Biosciences, Freiburg, Germany) for 1 hour at 37° C., washed, and diluted to 104 to 5*10⁴ cells per ml to obtain 10³ to 5*10³ target cells per well. CTL's were added in various effector:target (E:T) ratios and incubated for 4 hours. Supernatants were harvested and transferred to solid scintillator containing microplates (Perkin Elmer, Boston Mass., USA) and counted on a Topcount counter (Perkin Elmer). HPLC purified natural peptides or diluted synthetic peptides were tested for reactivity by loading ⁵¹Cr labelled T2 or donor EBV LCL cells for 1-2 hours at 37° C. and 5% CO₂ prior to addition of CTL.

### IFN-γ Secretion Assays

Quantification of CTL stimulation was performed by IFN-γ secretion assays. CTL's were cocultivated with various PBMNC cell populations or transfected Hela/A2 cells. 10⁵ Stimulator cells and 10⁴ CTL's were diluted in IMDM supplemented with 10% FBS and cultured in 96-well microtiterplates for 24 h. Supernatant was harvested and IFN-γ was measured by standard ELISA (Sanquin, Amsterdam, The Netherlands).

### Peptide Isolation, Purification and Characterization

Purification of peptides from EBV LCL recognized by RDR2 was performed as described before (den Haan et al. 1995, Science 268:1476-1480; Heemskerk et al. 2001, PNAS 98: 6806-6811). Briefly, frozen cell pellets were lysed using NP40 (Pierce, Rockford, USA) as a detergent. After high speed centrifugation supernatants were precleared by tumbling with CL4B sepharose beads(Amersham Biosciences, Uppsala, Sweden) and subsequent centrifugation. Supernatant was passed through affinity columns consisting of BB7.2 HLA-A2 antibodies covalently coupled to protein-A beads (Amersham Biosciences). HLA-A2 peptide complexes were eluted and disintegrated by 10% acetic acid. Peptides were separated from HLA-A2 monomers and β2-microglobulin by centrifugation through 5 kD filters (Vivascience, Hannover, Germany). Peptide containing filtrate was freeze dried. Peptide concentrates were dissolved in H₂O containing 0.1% TFA and injected on a Smart System (Amersham Biosciences) and subjected to RP-HPLC on a 10 cm×2.1 mm C2/C18 3 μm particle column at 0.2 ml/min. A gradient from 20% to 50% organic phase containing 0.1% TFA was run while 0.1 ml fractions were collected in siliconated vials and stored at −80° C. Isopropanol or Acetonitrile were used as organic phase. Fractions were tested for reactivity by loading a sample of 1-5 μl on ⁵¹Cr labelled T2 target cells prior to addition of the CTL. Selection of candidate peptides was performed by injecting samples on a 15 cm×75 μm Pepmap nano column of a LC system that was directly coupled to a Q-TOF1 mass spectrometer (Micromass, Manchester, UK). Subsequent peptide sequence analysis was performed by collision activated dissociation of selected masses on a HCT^{plus} mass spectrometer (Bruker Daltronics, Bremen, Germany).

### Sequence Analysis of the ADIR Gene

Since the ADIR gene was identified as the target for RDR2, patient and donor samples were analysed by sequence analysis. Trizol reagent (Invitrogen) was added to cell pellets and mRNA was isolated, purified and 4 μg was reverse transcribed into cDNA for 1 hour at 37° C. using M-MLV reverse transcriptase (Invitrogen) in accordance to manufacturers instructions. PCR reactions of nt 1-327 of the ADIR gene were performed in 50 ul GeneAmpII PCR buffer containing 1.5 mM MgCl₂, 250 μM dNTP's, 800 nM forward primer (5′-CTAGGCCGGCAGCCGGAT-3′), 800 nM reverse primer (5′-GCTGGCCCAACAGAGGAAG-3′), 2% DMSO and 1.5 U AmpliTaq DNA polymerase. Amplification on a Applied Biosystems GeneAmp PCR system 2400 was achieved following the program: 2′ 95° C., 35 cycli 15″ 95° C., 30″ 58° C., 1′ min 72° C., followed by a single elongation step of 7′ min at 72° C. Sequence reactions were performed on 1 μl of purified PCR product using the Big Dye Terminator v3.1 sequencing kit (Applied Biosystems, Foster City, Calif., USA) and 1 μM reverse primer following the program: 3′ 94° C., and 25 cycli 10″ 96° C., 5″ 58° C., 4′ 60° C. After DNA purification sequencing was performed using a ABI310 sequencer.

### Transfection of Constructs Containing the LB-ADIR-1F Epitope

Different constructs from donor and patient cDNA containing the ADIR gene were generated for transfection assays. PCR was performed on both patient and donor derived cDNA using 4 different forward primers and 1 reverse primer. Forward primers contained a flanking BgIII restriction site followed either directly NT 1-18 from the sequence start ^{TATAGATCTG}CTAGGCCGGCAGCCGGAT, or by a Kozak followed by the natural ATG from the normal ORF (5′-^{TATAGATCTGCCACC}ATGGTCCCGCAGCTC GGG-3′) or the natural ATG from the alternative ORF (5′-^{TATAGATCTGCCACC}ATGCTTCGC GGTCCGTG-3′). The reverse primer was chosen at nt 309-327 followed by a NotI restriction site (5′-^{TACGCGGCCGCTTA}GCTGGCCCAACAGAGGAAG-3′). PCR products were digested with restriction endonucleases Bglll (Roche, Mannheim, Germany) and NotI (Roche) in digestion buffer buffer H (Roche), purified using a PCR purification kit (Quiagen, Hilden, Germany), and ligated into previously generated BamH1 (Roche) and NotI sites of pCR3.1 expression vector (Invitrogen) using Rapid DNA ligation kit (Roche). Ligated vectors were used to transform competent E. coli and plated on ampicillin containing LB agar plates. The next day growing colonies were picked and expanded in LB-broth containing ampicillin. Plasmids were purified using Qiaprep Spin Miniprep (Qiagen). Hela cells stably transduced with HLA-A2 were seeded at 2*10⁴ cells per well in flat bottom plates. After 24 h, 100 ng plasmid was pre-incubated with Lipofectamine 2000 reagent (Invitrogen, Carlsbad, Calif., USA) and used to transfect 2*10⁴ Hela/A2 cells in Optimem I medium (Invitrogen, Paisley, Scotland). After 24 h 10⁴ RDR2 cells were added and again after 24 h 50 μl of supernatant was harvested and tested for IFN-γ secretion in ELISA.

### Ex Vivo Detection of LB-ADIR-1F Specific T Cells

Recombinant biotinilated HLA A*0201 monomers were folded in the presence of β2-microbglobulin with peptide SVAPALALFPA or SVAPALALSPA. Streptavidin-PE and streptavidin-APC tetramers were produced with refolded complexes as described previously (Altman et al. 1996 Science 274: 94-96). Tetrameric complexes were used to stain thawed patient samples taken at the indicated time points post SCT and DLI. Cells were counterstained by CD8 APC (Caltag, Burlingame, Calif., USA) and analysed by flowcytometry. Tetramer positive events were single well FACS sorted, expanded and tested for cytotoxicity.

### TCRBV Analysis of LB-ADIR-1F Specific T Cells

TCRBV expression of cytotoxic clones was determined by staining with FITC conjugated monoclonal antibodies to TCRBV7 (Beckmann Coulter, Mijdrecht, The Netherlands). Sequences of the TCRBV were determined as described previously (Kloosterboer F M et al. Leukemia 2004; 18(4)) and TCR chains were named in accordance with the nomenclature described by Arden et al., Immunogenetics 1995, 42. Counterstaining of tetramer positive T cells in patient material was performed using the TCRBV7 FITC antibodies.

### Quantitative PCR

Quantitative real-time PCR analysis was performed as described previously (Mensink et al. 1998, Br J. Haematol. 102: 768-774). Briefly, total RNA was isolated from different cell populations using Trizol(Invitrogen) according to manufacturer's instructions. In order to normalize for variations in the procedures for mRNA isolation and cDNA synthesis, to each cell sample 0.5% mouse spleen cells were added. Random primed cDNA was synthesized from 1 μg mRNA using the first strand cDNA synthesis kit for RT-PCR(AMV) (Roche). Quantitative real-time PCR was performed on an ABI/PRISM 7700 Sequence Detector System (Applied Biosystems) using qPCR Core Kit (Eurogentec, Seraing, Belgium). Human ADIR and PBGD results were normalized using the murine GAPDH expression and are depicted as gene expression per cell. Primers for hADIR were designed spanning exon 1 to 3:

forward primer
5′-GACGACTGTGACGAGGACGA-3′,
reverse primer
5′-CAAATGCTGGCCATGCAG-3′
and
probe
5′-(TET)-CTGGGCTGGCGCCTTCCTCTGT-(TAMRA)-3′.

Primers and probe for hPBGD were:

forward primer
5′-GCAATGCGGCTGCAA-3′,
reverse primer
5′-GGGTACCCACGCGAATG-3′
and
probe
5′-(TET)-CTCATCTTTGGGCTGTTTTCTTCCGCC-(TAMRA)-3′.

Primers and probe for mGAPDH were:

forward primer
5′-GGGCTCATGACCACAGTCCA-3′,
reverse primer
5′-ATACTTGGCAGGTTTCTCCAGG-3′
and
probe
5′-(TET)-TCCTACCCCCAATGTGTCCGTCGT-(TAMRA)-3′.

### Results

Isolation of an HLA-A2 Restricted CD8⁺ CTL Clone Recognizing a Frequently Expressed mHag.

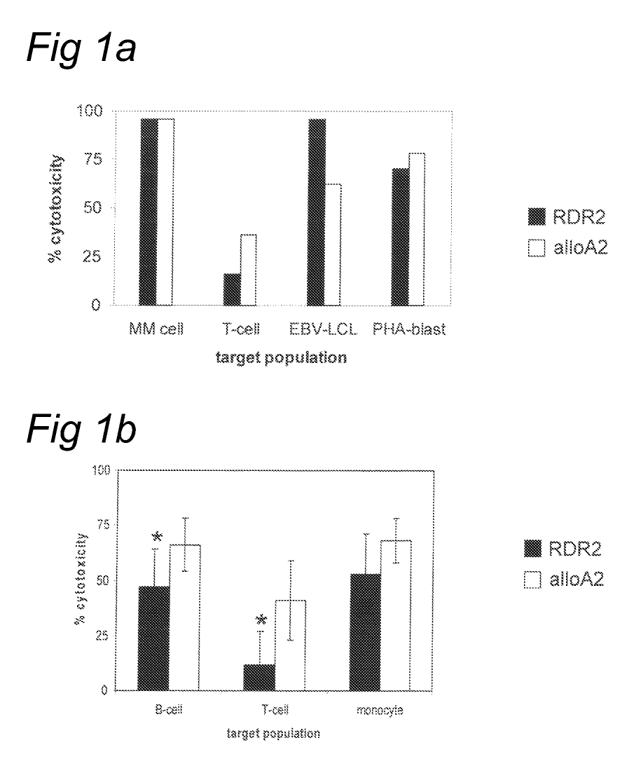

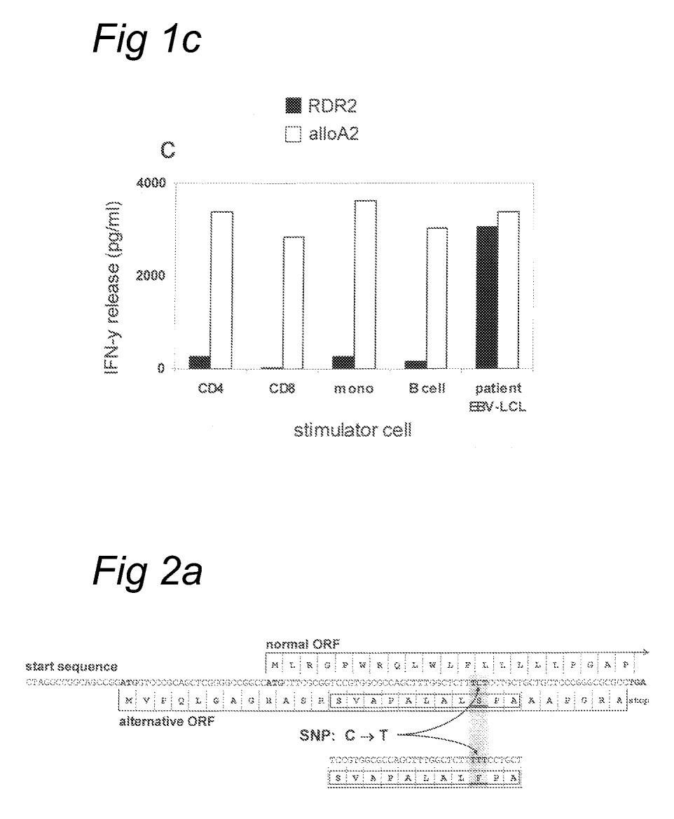

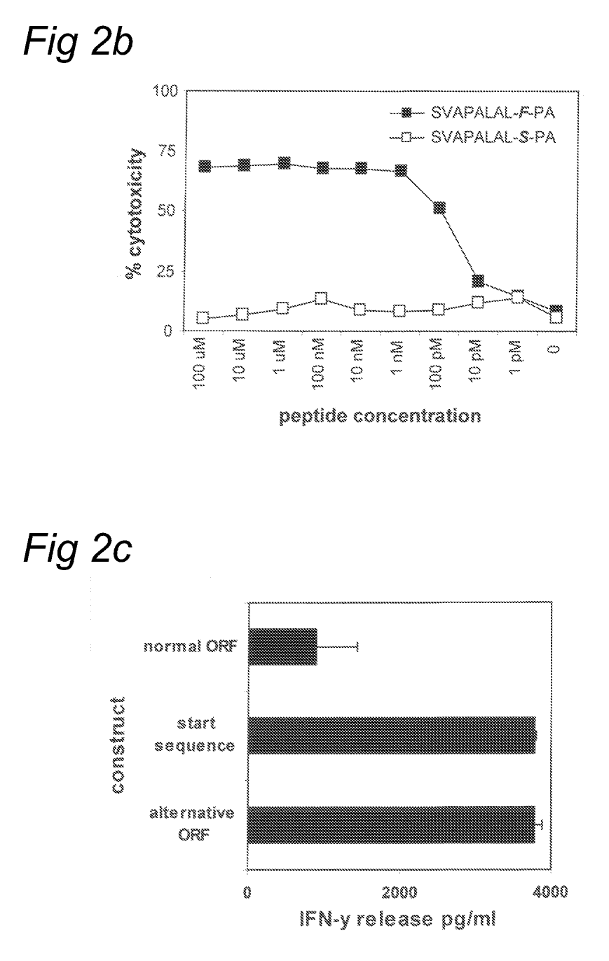

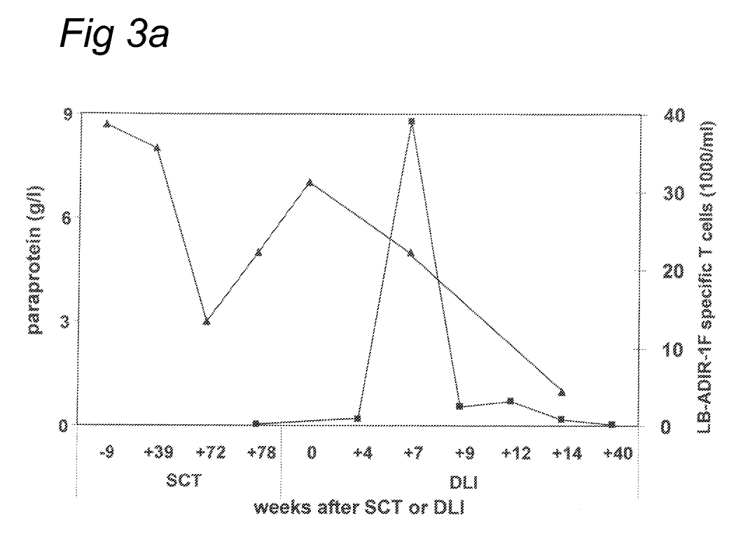

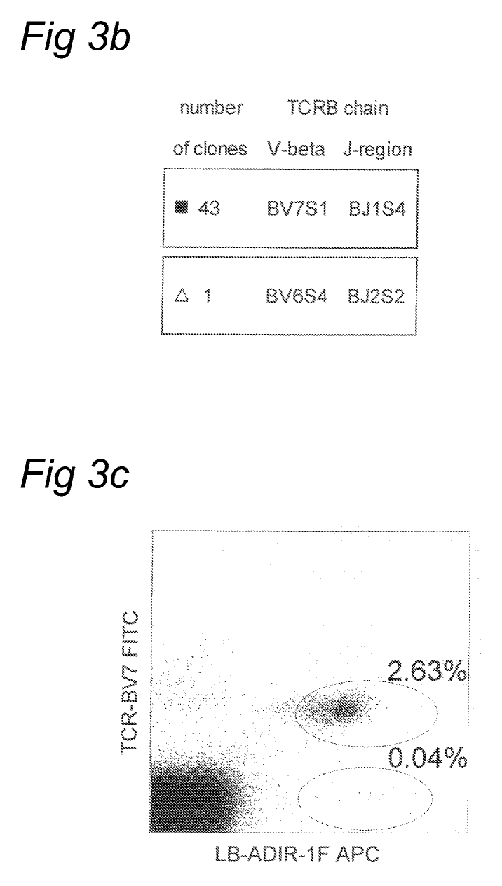

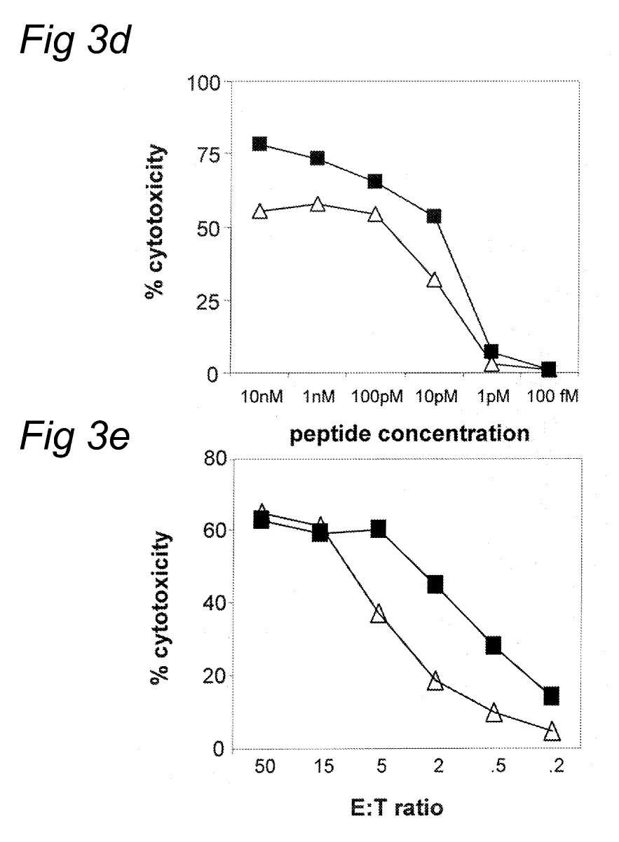

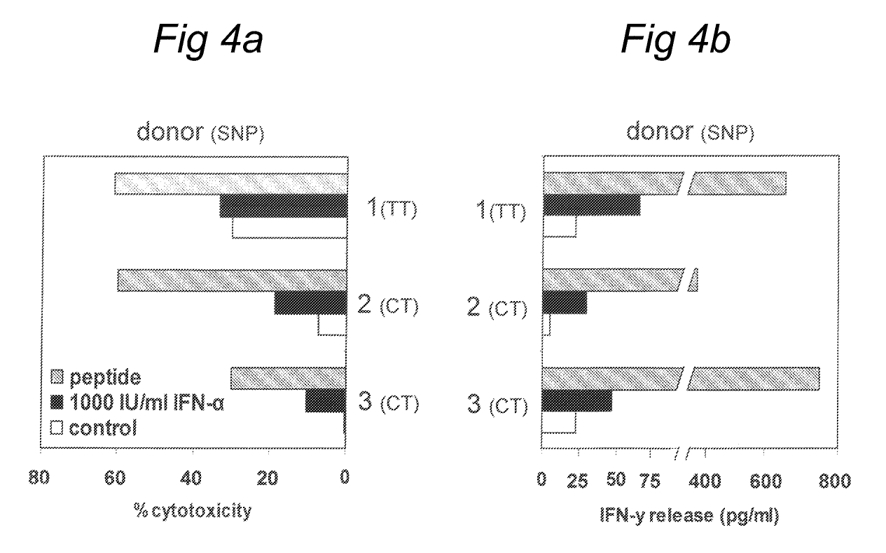

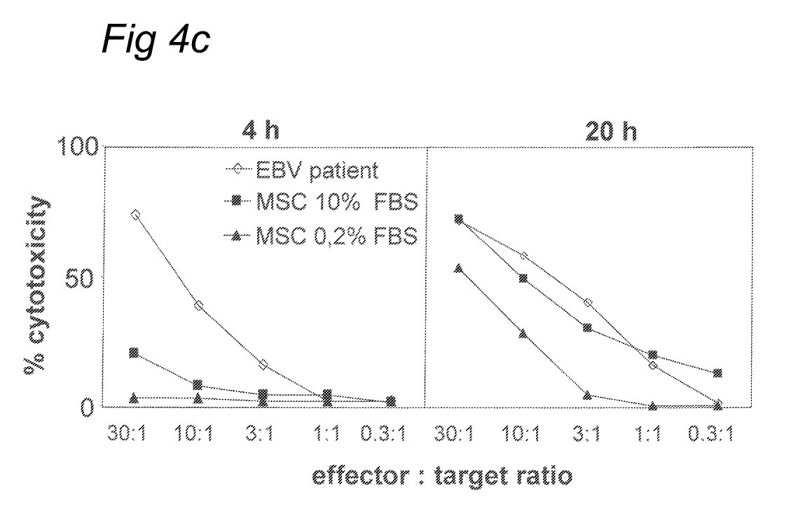

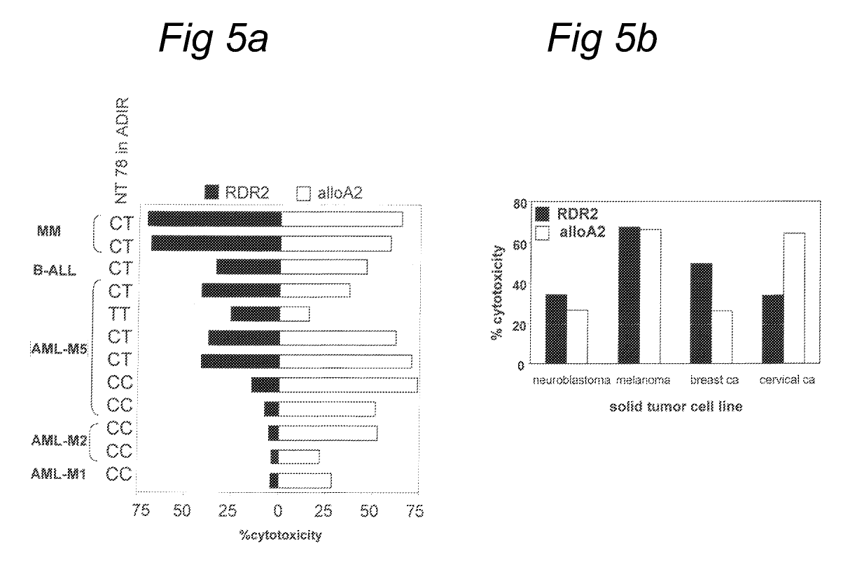

We previously described the isolation of various T cell clones from a female patient who was successfully treated with DLI after relapsed MM. CTL clones were generated by direct cloning of IFN-γ producing cells upon stimulation by irradiated bone marrow cells harvested from the patient prior to SCT. Panel studies using unrelated EBV LCL and blocking studies with HLA allele specific antibodies showed that recognition by the most dominant CTL clones was restricted by HLA-A2. Extensive panel studies using PHA blasts and EBV LCL of unrelated sibling pairs demonstrated that the majority of HLA-A2 restricted T cell clones designated RDR2, displayed an identical recognition pattern and lysed 57% of targets from all HLA-A2 individuals tested. All RDR2 clones were found to express an identical TCR BV7S1, N region and BJ1S4, illustrating that they were derived from the same clonal T cell (FM Kloosterboer et al. Leukemia. 2005 January; 19(1)). Since RDR2 was isolated from a MM patient we investigated the sensitivity of her MM cells to lysis by the T cell clone. The CFSE based cytotoxicity assay was performed to allow quantitative measurement of lysis of MM cells that were present in relatively low frequencies within the heterogeneous bone marrow sample from the patient (I Jedema et al, Blood. 2004, 103(7)). Lysis was measured after coculturing bone marrow cells with effector cells for 4 h in a 1:1 effector to target ratio. CD138 was used as a marker for the malignant MM cells and CD3 was used as a marker for non-malignant patient derived T cells. FIG. 1a illustrates that MM cells from the patient were strongly lysed whereas lysis of normal unstimulated T cells was low. Activated T cells (PHA blasts) and EBV LCL from the patient were strongly recognized. To further study susceptibility of normal hematopoietic cells to lysis by RDR2, sensitivity of mononuclear cell subpopulations from HLA-A2 positive mHag positive donors to recognition by RDR2 as compared to an alloA2 clone was analyzed. Whereas similar recognition by the alloA2 clone and RDR2 of PHA blasts and EVB LCL was observed, RDR2 mediated lysis of normal B and T cells was lower as compared to alloA2 mediated lysis (FIG. 1b). In addition, mononuclear cells were separated by magnetic bead cell sorting into CD4+ T-cells, CD8+ T-cells, monocytes and B-cells and were used to stimulate RDR2 and alloA2 CTL in IFN-γ release assays. Whereas all stimulator cell subpopulations were equally able to induce IFN-γ secretion by alloA2 CTL, stimulation of RDR2 was more than 10 fold lower (FIG. 1c).

In conclusion, RDR2 recognized an HLA-A2 restricted epitope causing strong lysis of multiple myeloma cells and activated T cells and B cells. In contrast, reactivity with normal non activated hematopoietic cells was relatively low as measured both by direct cytotoxicity and by interferon-γ secretion.

### Purification and Mass Spectrometric Identification of the Peptide

To identify the epitope that was recognized by CTL clone RDR2, 8×10¹⁰ EBV LCL cells expressing the antigen were lysed. Peptide-HLA complexes were affinity purified on a protein A column to which HLA-A2 specific BB7.2 antibodies were coupled. After elution and disintegration of peptide-HLA complexes with 10% acetic acid, 5 kD size centrifugation was performed to separate peptides from HLA-monomers and β2-microglobulin. After freeze drying the peptide mixture was subjected to RP-HPLC using isopropanol as organic solvent and fractions were collected. ⁵¹Cr labelled T2 cells were loaded with a small sample of each fraction. RDR2 was added and a single positive fraction could be detected. This fraction was subsequently subjected to RP-HPLC with acetonitrile as organic solvent and fractionated. Fractions were tested for reactivity and again a positive fraction was found. To determine the most abundant masses present in this fraction, part of the fraction was injected on a nano LC system directly coupled to a Q-TOF1 mass spectrometer. Abundantly present masses were fragmented by collision activated dissociation on a HCT^{plus} mass spectrometer. Analysis of obtained fragmentation patterns led to the sequence of several candidate peptides which were subsequently synthesized. Leucine and isoleucine are indistinguishable in fragmentation spectra and therefore, when present in a candidate peptide, a mixture of both aminoacids was used in the synthesis reaction at each position leucine or isoleucin was to be incorporated, leading to mixtures of peptides with either leucine or isoleucine at the desired position depicted as ‘X’. Synthetic peptides were used to load ⁵¹Cr labelled T2 cells. Lysis by RDR2 was reconstituted by a [M+2H]⁺⁺ candidate peptide with m/z=528.8 and sequence SVAPAXAXFPA at levels as low as 10 pM (data not shown). Furthermore, synthetic peptide SVAPAXAXFPA was subjected to fragmentation by collision activated dissociation mass spectrometry and yielded a fragmentation pattern identical to the eluted peptide (data not shown).

### Identification of a Polymorphic Gene Responsible for RDR2 Recognition

A blast search of sequence SVAPAXAXFPA against a six-frame translation of the EMBL nucleotide database revealed 100% identity to amino acids 13-23 SVAPALALFPA from an alternative ORF of the ADIR gene, also known as TOR3A (Dron et al. 2002, Genomics 79: 315-325). A known single nucleotide polymorphism (SNP) in ADIR at nucleotide 78 from C to T results in an amino acid residue change in an alternative transcript from serine(S) into a phenylalanine(F) at position 21, corresponding to position 9 of the eluted peptide (FIG. 2a). Both peptides were synthesized and loaded on T2 cells. Peptide SVAPALAL-F-PA but not SVAPALAL-S-PA was recognized by RDR2 (FIG. 2b). RNA from patient and donor cells was reverse-transcribed to cDNA and amplified using primers flanking the SNP resulting in a 327 nt fragment. Sequence analysis of this fragment revealed that the donor was CC homozygous and the patient CT heterozygous. To demonstrate that patient type polymorphism T but not donor type C C of this gene was responsible for recognition by RDR2, constructs were generated from both donor and patient. Since RDR2 recognized a peptide arising from an alternative ORF controlled by a start codon 5′ upstream from the normal start codon, 3 different forward primers were composed. The first primer was chosen at the normal start codon, thus lacking the alternative start codon. The second primer was chosen at the start of the transcript, thus providing both natural start codons. The third primer was chosen at the alternative start codon and also contained the normal start codon. Apart from the second primer all other primers contained Kozak sequences next to the ATG start codons. The constructs were transiently transfected into Hela cells stably transduced with a LZRS vector containing HLA-A*0201 and reporter gene NGF-receptor. Patient-derived constructs induced IFN-γ release by RDR2 CTL. Furthermore, transfection of constructs containing only the normal ORF startcodon and lacking the alternative ORF startcodon showed a strong decrease of CTL recognition (FIG. 2c). All donor derived constructs failed to induce INF-γ by RDR2 (data not shown). Next, a panel of 74 unrelated HLA-A2 positive individuals was analysed by sequencing for determination of the polymorphism, and susceptibility to lysis of PHA blasts by RDR2. A 100% correlation between presence of this specific SNP and CTL reactivity proved that the SNP of C to T at nt 78 in the ADIR gene generates the mHag epitope SVAPALALFPA that is recognized by RDR2 (Table 1). This mHag was designated LB-ADIR-1F.

TABLE 1
Correlation of SNP and CTL reactivity in 76 individuals

| | Number of | Lysis of PHA | |
|---|---|---|---|
| ADIR nt 78¹ | individuals | blasts | frequency (%) |
| CC | 33 | 0/33 | 43 |
| CT | 36 | 36/36 | 47 |
| TT | 7 | 7/7 | 9 |

¹Sequence analysis of nt 1-327 of the ADIR gene, nucleotide 78 represents the SNP.

Tetramer Staining and FACS Sorting of LB-ADIR-1F specific CTL's

Both peptide LB-ADIR-1F and peptide SVAPALAL-S-PA were able to bind to recombinant HLA A*0201 molecules and tetrameric complexes were produced. RDR2 specifically bound the LB-ADIR-1F tetramer whereas control SVAPALAL-S-PA tetramers and irrelevant HA-1control tetramers were negative (data not shown). LB-ADIR-1F tetramers were used to analyse a series of blood samples that were taken from the patient before and after DLI. Serum paraprotein levels were analysed as a marker for disease activity. Whereas LB-ADIR-1F specific T cells were not detectable prior to DLI, at 7 weeks post DLI high numbers of LB-ADIR-1F specific CD8+ T cells could be detected (FIG. 3a). The appearance of LB-ADIR-1F specific T cells coincided with development of acute GVHD grade UI and complete remission. GVHD was treated successfully with 1 mg prednisolone/kg body weight and cyclosporin A. Tetramer positive T cells were clonally isolated by FACS sorting and expanded. All tetramer positive CTL clones were able to lyse both patient EBV cells and LB-ADIR-1F pulsed donor EBV cells (data not shown). TCR characterization of RDR2 showed usage of V-beta BV7S1 and J-region BJiS4 in 43 out of 44 growing clones analysed. One clone, however, expressed TCRBV6S4 (FIG. 3b). Analysis of the patient patient sample at 7 weeks post DLI revealed that a low percentage of LB-ADIR-1F positive T cells did not stain with antibodies directed against TCR-BV7 (FIG. 3C). Functional comparing of the original RDR2, newly isolated identical TCR BV7S1 clones and TCR BV6S4 expressing clones was performed. Whereas cytotoxicity of the original RDR2 and newly isolated BV7S1 clones was similar (data not shown), the BV6S4 clone clearly showed diminished recognition of peptide loaded T2 cells (FIG. 3d). When HLA-A2 positive and LB-ADIR-1F expressing EBV LCL cells were used as target cells, again the BV6S4 clone displayed lower cytotoxicity (FIG. 3e). Similar results were obtained using PHA blasts (data not shown).

### ADIR Gene Expression and Modulation of Recognition

Previous studies on the ADIR gene have indicated that IFN-α could enhance gene expression (Dron et al., 2002, supra). Therefore, the effect of IFN-α on LB-ADIR-1F recognition by RDR2 was studied using MNC of LB-ADIR-1F expressing donors. MNC were precultured for 48 hours in the absence or presence of 1000 IU/ml IFN-α prior to addition of CTL RDR2 at a 1:1 ratio. Maximal recognition was determined by testing MNC pulsed with saturating concentrations of synthetic peptide. Cytotoxicity was measured in a 4 h CFSE-assay (FIG. 4a) and IFN-γ release was measured after 24 h (FIG. 4b). INF-α enhanced both susceptibility to lysis and stimulatory capacity. LB-ADIR-1F expressing mesenchymal stem cells and EBV LCL were used as target cells in cytotoxicity assays. RDR2 lysis of both active MSC continuously cultured in 10% FBS and resting MSC precultured for 48 h in 0.2% FBS was measured after 4 and 20 h (FIG. 4c). Strong lysis of EBV LCL was observed after 4 h, whereas lysis of active MSC was low. Resting MSC were not lysed by RDR2. Prolonged incubation resulted in comparable lysis of both EBV LCL and active MSC whereas resting MSC still showed decreased susceptibility to RDR2 lysis. ADIR gene expression was measured by performing quantitative PCR. To each cell sample a fixed percentage of 0.5% murine spleen cells was added. Each sample was assayed for expression of ADIR, PBGD and murine GAPDH. In order to exclude variation in mRNA isolation and cDNA synthesis both ADIR and PBGD expression levels were normalized to the murine GAPDH expression level. In resting cells mRNA levels of both ADIR and PBGD were low as compared to levels in cultured PHA blasts, EBV LCL and MSC indicating an overall increase in gene expression due to activation and culture conditions (Table 2). In addition, freshly isolated donor MNC were incubated with 500 IU/ml IFN-α for 24 and 48 h prior to harvesting. An IFN-α dependent increase in ADIR mRNA expression was observed (Table 3). In conclusion, we show that both LB-ADIR-1F antigen and ADIR gene expression is relatively low under steady-state conditions and can be strongly upregulated during activation of the cell.

TABLE 2
ADIR gene expression in different cell types

Relative gene expression level per cell ²

| cell type | n | ADIR | PBGD |
|---|---|---|---|
| MNC | 5 | 84 ± 16 | 48 ± 8 |
| PHA blast | 5 | 5084 ± 3637 | 2824 ± 1323 |
| EBV LCL | 3 | 2250 ± 385 | 1766 ± 464 |
| MSC | 2 | 5268 ± 710 | 8456 ± 460 |

² Prior to mRNA isolation and cDNA synthesis 0.5% mouse spleen cells were added to each sample. Quantitative PCR data were normalized to murine GAPDH.

TABLE 3
IFN-α modulation of ADIR gene expression

| | relative gene expression per cell ² | |
|---|---|---|
| incubation | ADIR | PBGD |

| time (h) | — | IFN-a ³ | — | IFN-a ³ |
|---|---|---|---|---|
| 0 | 68 | 68 | 16 | 16 |
| 24 | 155 | 538 | 49 | 45 |
| 48 | 268 | 631 | 55 | 80 |

² Prior to mRNA isolation and cDNA synthesis 0.5% mouse spleen cells were added to each sample. Quantitative PCR data were normalized to murine GAPDH.
³ Cells were incubated in the absence or presence of 500 IU/ml INFα.

### Expression of LB-ADIR-1F on MM Cells, Leukemic Blasts and Solid Tumor Cell Lines

To investigate applicability of LB-ADIR-1F as a target for immunotherapy we investigated reactivity of RDR2 on hematological malignancies. A panel of HLA-A2 positive MM and leukemic cells was subjected to RDR2 lysis and sequence analysis on the LB-ADIR-1F polymorphism. Most prominently recognition of MM was found, but also leukemic cells expressing the LB-ADIR-1F SNP could be lysed (FIG. 5a). A panel of SNP positive HLA-A2 expressing solid tumor lines was tested for susceptibility to lysis by RDR2. Melanoma BROWN, cervical carcinoma CASK1, breast carcinoma MCF-7 and neuroblastoma TT could all be lysed by RDR2 at levels comparable to lysis by the alloA2 specific control clone (FIG. 5b).

### REFERENCE LIST

(1) Appelbaum F R. The current status of hematopoietic cell transplantation. Annu Rev Med. 2003; 54:491-512.
(2) Thomas E D. Karnofsky Memorial Lecture. Marrow transplantation for malignant diseases. J Clin Oncol. 1983; 1:517-531.
(3) Horowitz M M, Gale R P, Sondel P M et al. Graft-versus-leukemia reactions after bone marrow transplantation. Blood. 1990; 75:555-562.
(4) Faber L M, Luxemburg-Heijs S A, Willemze R, Falkenburg J H. Generation of leukemia-reactive cytotoxic T lymphocyte clones from the HLA-identical bone marrow donor of a patient with leukemia. J Exp Med. 1992; 176:1283-1289.
(5) Niederwieser D, Grassegger A, Aubock J et al. Correlation of minor histocompatibility antigen-specific cytotoxic T lymphocytes with graft-versus-host disease status and analyses of tissue distribution of their target antigens.

Blood. 1993; 81:2200-2208.
(6) Apperley J F, Jones L, Hale G et al. Bone marrow transplantation for patients with chronic myeloid leukaemia: T-cell depletion with Campath-1 reduces the incidence of graft-versus-host disease but may increase the risk of leukaemic relapse. Bone Marrow Transplant. 1986; 1:53-66.
(7) Marmont A M, Horowitz M M, Gale R P et al. T-cell depletion of HLA-identical transplants in leukemia. Blood. 1991; 78:2120-2130.
(8) Collins R H, Jr., Shpilberg O, Drobyski W R et al. Donor leukocyte infusions in 140 patients with relapsed malignancy after allogeneic bone marrow transplantation. J Clin Oncol. 1997; 15:433-444.
(9) Kolb H J, Schattenberg A, Goldman J M et al. Graft-versus-leukemia effect of donor lymphocyte transfusions in marrow grafted patients. Blood. 1995; 86:2041-2050.
(10) Falkenburg J H F, van de Corput L, Marijt E W A, Willemze R. Minor histocompatibility antigens in human stem cell transplantation. Experimental Hematology. 2003; 31:743-751.
(11) Goulmy E. Human minor histocompatibility antigens. Curr Opin Immunol. 1996; 8:75-81.
(12) Brickner A G, Warren E H, Caldwell J A et al. The immunogenicity of a new human minor histocompatibility antigen results from differential antigen processing. J Exp Med. 2001; 193:195-206.
(13) Spierings E, Brickner A G, Caldwell J A et al. The minor histocompatibility antigen HA-3 arises from differential proteasome-mediated cleavage of the lymphoid blast crisis (Lbc) oncoprotein. Blood. 2003; 102:621-629.
(14) Akatsuka Y, Nishida T, Kondo E et al. Identification of a polymorphic gene, BCL2A1, encoding two novel hematopoietic lineage-specific minor histocompatibility antigens. J Exp Med. 2003; 197:1489-1500.
(15) den Haan J M, Sherman N E, Blokland E et al. Identification of a graft versus host disease-associated human minor histocompatibility antigen. Science. 1995; 268: 1476-1480.
(16) den Haan J M, Meadows L M, Wang W et al. The minor histocompatibility antigen HA-1: a diallelic gene with a single amino acid polymorphism. Science. 1998; 279:1054-1057.
(17) Dolstra H, Fredrix H, Maas F et al. A human minor histocompatibility antigen specific for B cell acute lymphoblastic leukemia. J Exp Med. 1999; 189:301-308.
(18) van der Harst D, Goulmy E, Falkenburg J H et al. Recognition of minor histocompatibility antigens on lymphocytic and myeloid leukemic cells by cytotoxic T-cell clones. Blood. 1994; 83:1060-1066.
(19) Bleakley M, Riddell S R. Molecules and mechanisms of the graft-versus-leukaemia effect. Nat Rev Cancer. 2004; 4:371-380.
(20) Denhaan J M M, Sherman N E, Blokland E et al. Identification of A Graft-Versus-Host Disease-Associated Human Minor Histocompatibility Antigen.

Science. 1995; 268:1476-1480.
(21) Pierce R A, Field E D, Mutis T et al. The HA-2 minor histocompatibility antigen is derived from a diallelic gene encoding a novel human class I myosin protein. J. Immunol. 2001; 167:3223-3230.
(22) Mocellin S, Mandruzzato S, Bronte V, Lise M, Nitti D. Part I: Vaccines for solid tumours. Lancet Oncol. 2004; 5:681-689.
(23) Rosenberg S A, Yang J C, Restifo N P. Cancer immunotherapy: moving beyond current vaccines. Nat. Med. 2004; 10:909-915.
(24) Molldrem J J, Clave E, Jiang Y Z et al. Cytotoxic T lymphocytes specific for a nonpolymorphic proteinase 3 peptide preferentially inhibit chronic myeloid leukemia colony-forming units. Blood. 1997; 90:2529-2534.
(25) Vogt M H, de Paus R A, Voogt P J, Willemze R, Falkenburg J H. DFFRY codes for a new human male-specific minor transplantation antigen involved in bone marrow graft rejection. Blood. 2000; 95:1100-1105.
(26) Vogt M H, Goulmy E, Kloosterboer F M et al. UTY gene codes for an HLA-B60-restricted human male-specific minor histocompatibility antigen involved in stem cell graft rejection: characterization of the critical polymorphic amino acid residues for T-cell recognition. Blood. 2000; 96:3126-3132.
(27) Vogt M H, van den Muijsenberg J W, Goulmy E et al. The DBY gene codes for an HLA-DQ5-restricted human male-specific minor histocompatibility antigen involved in graft-versus-host disease. Blood. 2002; 99:3027-3032.
(28) Meadows L, Wang W, den Haan J M et al. The HLA-A*0201-restricted H—Y antigen contains a posttranslationally modified cysteine that significantly affects T cell recognition. Immunity. 1997; 6:273-281.
(29) Wang W, Meadows L R, den Haan J M et al. Human H—Y: a male-specific histocompatibility antigen derived from the SMCY protein. Science. 1995; 269:1588-1590.
(30) Warren E H, Gavin M A, Simpson E et al. The human UTY gene encodes a novel HLA-BS-restricted H—Y antigen. Journal of Immunology. 2000; 164:2807-2814.
(31) Torikai H, Akatsuka Y, Miyazaki M et al. A novel HLA-A*3303-restricted minor histocompatibility antigen encoded by an unconventional open reading frame of human TMSB4Y gene. J. Immunol. 2004; 173:7046-7054.
(32) Murata M, Warren E H, Riddell S R. A human minor histocompatibility antigen resulting from differential expression due to a gene deletion. J Exp Med. 2003; 197:1279-1289.
(33) Dron M, Meritet J F, Dandoy-Dron F et al. Molecular cloning of ADIR, a novel interferon responsive gene encoding a protein related to the torsins. Genomics. 2002; 79:315-325.

1-18. (canceled)

19. A peptide or a cell for use in the treatment of a subject suffering from a malignancy expressing the hADIR gene,
wherein
a) the peptide comprises an amino acid sequence encoded by an open reading frame as present in the nucleotide sequence of a transcript of a naturally occurring hADIR allele;
b) the amino acid sequence of a) comprises a polymorphic MHC class I or II minor histocompatibility binding peptide; and
c) the nucleic acid sequence encoding the peptide comprises a single nucleotide polymorphism (SNP) at position 78 of hADIR or another SNP of TABLE A;
or wherein the cell is
i) a T lymphocyte comprising a T cell receptor capable of interacting with the MHC binding peptide of b); or
ii) a host cell comprising the nucleic acid molecule encoding the T cell receptor as defined in i), optionally comprised within a nucleic acid vector and optionally displaying the TCR.

20. A peptide or a cell according to claim 19, wherein the open reading frame of a) is selected from the amino acid sequences of SEQ ID NO: 3-5.

21. A peptide or a cell according to claim 19 or claim 20, wherein the MHC class I or II minor histocompatibility binding peptide is in the context of an MHC class I or MHC class II molecule.

22. A peptide or a cell according to any one of claims 19 to 21 wherein the subject has undergone an allogeneic hematopoietic stem cell transplantation.

23. A peptide or a cell according to any one of claims 19 to 22, wherein the malignancy is a hematopoietic malignancy.

24. A peptide or a cell according to any one of claims 19 to 22, wherein the malignancy is a solid tumor present in or originating from a dispensible organ or tissue.

25. A peptide or a cell according to claim 24, wherein the dispensible tissue or organ is selected from the group consisting of bone marrow, spleen, testes, kidneys, ovaria, breast, prostate, thyroid, cervix, uterus and pancreas.

26. A peptide comprising an amino acid sequence encoded by an open reading frame as present in the nucleotide sequence of a transcript of a naturally occurring hADIR allele, wherein the amino acid sequence comprises a polymorphic MHC class I or II minor histocompatibility binding peptide and wherein the nucleic acid sequence encoding the peptide comprises a single nucleotide polymorphism (SNP) at position 78 of hADIR or another SNP of TABLE A.

27. A peptide according to claim 26, wherein the open reading frame of a) is selected from the amino acid sequences of SEQ ID NO: 3-5.

28. A nucleic acid molecule encoding the peptide according to claim 26 or claim 27.

29. A TCR receptor capable of interacting with the MHC binding peptide of claim 26.

30. A nucleic acid molecule encoding the TCR receptor of claim 29, optionally comprised within a nucleic acid vector.

31. A T lymphocyte comprising a T cell receptor capable of interacting with the MHC binding peptide of claim 26.

32. A host cell comprising the nucleic acid molecule encoding the T cell receptor as defined in claim 29, optionally comprised within a nucleic acid vector and optionally displaying the TCR.

33. A pharmaceutical composition comprising at least one of:
i) a peptide as defined in claim 26 or claim 27;
ii) a cell as defined in claims 31 or 32;
iii) a nucleic acid molecule and/or a vector encoding the peptide as defined in claim 26 or claim 27;
iv) a gene and/or a vector encoding a TCR as defined in claim 29;
and at least one pharmaceutically acceptable excipient.

34. A human or humanized antibody specific for a polymorphic hADIR minor histocompatibility antigen, the antigen optionally being in the context of an HLA molecule.

35. The antibody according to claim 34, capable of binding an hADIR mHag encoded by a hADIR nucleotide sequence comprising a SNP at nucleotides 78, 672, 740, 752, 856, 1454, 1761 and/or 2011 in SEQ ID NO: 1, optionally in context of MHC class I or MHC class II molecule.


SEQUENCE LISTING (5 sequences)

SEQ ID NO: 1 - DNA, 2081 bp, unknown
  human ADIR cDNA (locus NM_022371), also known as Homo sapiens torsin family 3, member A (TOR3A)
ctaggccggcagccggatggtcccgcagctcggggccggccatgcttcgcggtccgtggcgccagctttggctctttctcctgctgctgctcccgggcgcgcctgagccccgcggcgcctccaggccgtgggagggaaccgacgagccgggctcggcctgggcctggccgggcttccagcgcctgcaggagcagctcagggcggcgggtgccctctccaagcggtactggacgctcttcagctgccaggtgtggcccgacgactgtgacgaggacgaggaggcagccacggggcccctgggctggcgccttcctctgttgggccagcggtacctggacctcctgaccacgtggtactgcagcttcaaagactgctgccctagaggggattgcagaatctccaacaactttacaggcttagagtgggacctgaatgtgcggctgcatggccagcatttggtccagcagctggtcctaagaacagtgaggggctacttagagacgccccagccagaaaaggcccttgctctgtcgttccacggctggtctggcacaggcaagaacttcgtggcacggatgctggtggagaacctgtatcgggacgggctgatgagtgactgtgtcaggatgttcatcgccacgttccactttcctcaccccaaatatgtggacctgtacaaggagcagctgatgagccagatccgggagacgcagcagctctgccaccagaccctgttcatcttcgatgaagcggagaagctgcacccagggctgctggaggtccttgggccacacttagaacgccgggcccctgagggccacagggctgagtctccatggactatctttctgtttctcagtaatctcaggggcgatataatcaatgaggtggtcctaaagttgctcaaggctggatggtcccgggaagaaattacgatggaacacctggagccccacctccaggcggagattgtggagaccatagacaatggctttggccacagccgtcttgtgaaggaaaacctgattgactacttcatccccttcctgcctttggagtaccgtcacgtgaggctgtgtgcacgggatgccttcctgagccaggagctcctgtataaagaagagacactggatgaaatagcccagatgatggtgtatgtccccaaggaggaacaactcttttcttcccagggctgcaagtctatttcccagaggattaactacttcctgtcatgaaggctagaggaagacttcctggaactgcctttcttccactaacaggaccctgggacctgtaggagcaccccgtttgggactgtgaggtgtttgagggtgtggactggcatccagcagccactaacaaacacacaactggtgtgtaaaaggcaggccttacattagaagccaagccaatcctttttcttttttttggaggtcccaccgagatagatagataggaacttggattgctgaattcaaaaacagagcccattcttaagatcacttggtgccttaaagacacgcattccaaagtggaatgtggttgaagaaagtgggccaggtggttgaagaaagccatgtgggagctcagcaaatcccaagggcttattatgacactccagatggtctccttagcatctcagctcttctgcaaggaagagcttgggtgttaggcctcagaggctgtagggtccttgggttacagagccggggagaacgaagttctgtgacccaggggtggagaatacactctaggtttgcgggctggtgggctttcaaattggtacttccagaggaaagccaagctgcttctgttgtgagcgaatcagccaagagcctgaggctgaagggaaaagtacacagaggaagatattttacaaaccaggtcagtgtaggccaagacttatggtctacagattttggcgggggaggggggaccttttcaaagacaatagggggtcttgacatgtttgttgtatgtaaagatgataagattaaaatttttgattttcctaaaaaaaaaaaaaaaaaaaaaaaaaaaaaaaaaaaaaaaaaaaaaaaaaaaaaaaaaaaaacctcggg

SEQ ID NO: 2 - protein, 397 aa, unknown
  human ADIR protein, normal reading frame (+3), depicted from translation start codon as shown in SEQ ID NO 1.
MLRGPWRQLWLFLLLLLPGAPEPRGASRPWEGTDEPGSAWAWPGFQRLQEQLRAAGALSKRYWTLFSCQVWPDDCDEDEEAATGPLGWRLPLLGQRYLDLLTTWYCSFKDCCPRGDCRISNNFTGLEWDLNVRLHGQHLVQQLVLRTVRGYLETPQPEKALALSFHGWSGTGKNFVARMLVENLYRDGLMSDCVRMFIATFHFPHPKYVDLYKEQLMSQIRETQQLCHQTLFIFDEAEKLHPGLLEVLGPHLERRAPEGHRAESPWTIFLFLSNLRGDIINEVVLKLLKAGWSREEITMEHLEPHLQAEIVETIDNGFGHSRLVKENLIDYFIPFLPLEYRHVRLCARDAFLSQELLYKEETLDEIAQMMVYVPKEEQLFSSQGCKSISQRINYFLS

SEQ ID NO: 3 - protein, 692 aa, unknown
  normal reading frame (+3) of hADIR/TOR3A gene; including amino acids preceding the translation start codon as shown in SEQ ID NO 1; X represents stop codonds
RPAAGWSRSSGPAMLRGPWRQLWLFLLLLLPGAPEPRGASRPWEGTDEPGSAWAWPGFQRLQEQLRAAGALSKRYWTLFSCQVWPDDCDEDEEAATGPLGWRLPLLGQRYLDLLTTWYCSFKDCCPRGDCRISNNFTGLEWDLNVRLHGQHLVQQLVLRTVRGYLETPQPEKALALSFHGWSGTGKNFVARMLVENLYRDGLMSDCVRMFIATFHFPHPKYVDLYKEQLMSQIRETQQLCHQTLFIFDEAEKLHPGLLEVLGPHLERRAPEGHRAESPWTIFLFLSNLRGDIINEVVLKLLKAGWSREEITMEHLEPHLQAEIVETIDNGFGHSRLVKENLIDYFIPFLPLEYRHVRLCARDAFLSQELLYKEETLDEIAQMMVYVPKEEQLFSSQGCKSISQRINYFLSXRLEEDFLELPFFHXQDPGTCRSTPFGTVRCLRVWTGIQQPLTNTQLVCKRQALHXKPSQSFFFFLEVPPRXIDRNLDCXIQKQSPFLRSLGALKTRIPKWNVVEESGPGGXRKPCGSSANPKGLLXHSRWSPXHLSSSARKSLGVRPQRLXGPWVTEPGRTKFCDPGVENTLXVCGLVGFQIGTSRGKPSCFCCERISQEPEAEGKSTQRKIFYKPGQCRPRLMVYRFWRGRGDLFKDNRGSXHVCCMXRXXDXNFXFSXKKKKKKKKKKKKKKKKKKKNL

SEQ ID NO: 4 - protein, 692 aa, unknown
  alternative reading frame (+2) of hADIR/TOR3A; X represents stop codons
XAGSRMVPQLGAGHASRSVAPALALSPAAAPGRAXAPRRLQAVGGNRRAGLGLGLAGLPAPAGAAQGGGCPLQAVLDALQLPGVARRLXRGRGGSHGAPGLAPSSVGPAVPGPPDHVVLQLQRLLPXRGLQNLQQLYRLRVGPECAAAWPAFGPAAGPKNSEGLLRDAPARKGPCSVVPRLVWHRQELRGTDAGGEPVSGRADEXLCQDVHRHVPLSSPQICGPVQGAADEPDPGDAAALPPDPVHLRXSGEAAPRAAGGPWATLRTPGPXGPQGXVSMDYLSVSQXSQGRYNQXGGPKVAQGWMVPGRNYDGTPGAPPPGGDCGDHRQWLWPQPSCEGKPDXLLHPLPAFGVPSREAVCTGCLPEPGAPVXRRDTGXNSPDDGVCPQGGTTLFFPGLQVYFPEDXLLPVMKARGRLPGTAFLPLTGPWDLXEHPVWDCEVFEGVDWHPAATNKHTTGVXKAGLTLEAKPILFLFFGGPTEIDRXELGLLNSKTEPILKITWCLKDTHSKVECGXRKWARWLKKAMWELSKSQGLIMTLQMVSLASQLFCKEELGCXASEAVGSLGYRAGENEVLXPRGGEYTLGLRAGGLSNWYFQRKAKLLLLXANQPRAXGXREKYTEEDILQTRSVXAKTYGLQILAGEGGPFQRQXGVLTCLLYVKMIRLKFLIFLKKKKKKKKKKKKKKKKKKKKP

SEQ ID NO: 5 - protein, 693 aa, unknown
  Alternative reading frame (+1) of hADIR/TOR3A; X represent stop codons
LGRQPDGPAARGRPCFAVRGASFGSFSCCCSRARLSPAAPPGRGREPTSRARPGPGRASSACRSSSGRRVPSPSGTGRSSAARCGPTTVTRTRRQPRGPWAGAFLCWASGTWTSXPRGTAASKTAALEGIAESPTTLQAXSGTXMCGCMASIWSSSWSXEQXGATXRRPSQKRPLLCRSTAGLAQARTSWHGCWWRTCIGTGXXVTVSGCSSPRSTFLTPNMWTCTRSSXXARSGRRSSSATRPCSSSMKRRSCTQGCWRSLGHTXNAGPLRATGLSLHGLSFCFSVISGAIXSMRWSXSCSRLDGPGKKLRWNTWSPTSRRRLWRPXTMALATAVLXRKTXLTTSSPSCLWSTVTXGCVHGMPSXARSSCIKKRHWMKXPRXWCMSPRRNNSFLPRAASLFPRGLTTSCHEGXRKTSWNCLSSTNRTLGPVGAPRLGLXGVXGCGLASSSHXQTHNWCVKGRPYIRSQANPFSFFWRSHRDRXIGTWIAEFKNRAHSXDHLVPXRHAFQSGMWLKKVGQVVEESHVGAQQIPRAYYDTPDGLLSISALLQGRAWVLGLRGCRVLGLQSRGERSSVTQGWRIHSRFAGWWAFKLVLPEESQAASVVSESAKSLRLKGKVHRGRYFTNQVSVGQDLWSTDFGGGGGTFSKTIGGLDMFVVCKDDKIKIFDFPKKKKKKKKKKKKKKKKKKKKTS